(19) United States
(12) Patent Application Publication
(10) Pub. No.: US 2024/0233874 A1
(43) Pub. Date: Jul. 11, 2024

(54) SYSTEM OF PREPROCESSORS TO HARMONIZE DISPARATE 'OMICS DATASETS BY ADDRESSING BIAS AND/OR BATCH EFFECTS

(71) Applicant: Genialis Inc., Boston, MA (US)

(72) Inventors: Miha Stajdohar, Ljubljana (SI); Matjaz Zganec, Ruse (SI); Robert Cvitkovic, Crnomelj (SI); Roman Lustrik, Ljubljana (SI); Luka Ausec, Kranj (SI); Rafael Rosengarten, San Ramon, CA (US); Daniel William Pointing, Sydney (AU)

(21) Appl. No.: 18/417,781

(22) Filed: Jan. 19, 2024

Related U.S. Application Data
(63) Continuation of application No. PCT/US2022/037860, filed on Jul. 21, 2022.
(60) Provisional application No. 63/224,210, filed on Jul. 21, 2021.

(51) Int. Cl. G16B 35/10; G16B 40/00; G16B 50/30

(52) U.S. Cl. CPC G16B 35/10; G16B 40/00; G16B 50/30

(57) ABSTRACT

Systems of preprocessors are provided to harmonize disparate ‘omics datasets by addressing bias and/or batch effects. Methods are provided for harmonization of datasets, for generation of libraries of preprocessors for use in harmonization, and for training classifiers leveraging harmonization.

### CROSS-REFERENCE TO RELATED APPLICATIONS

This application is a continuation of International Patent Application No. PCT/US2022/037860, filed Jul. 21, 2022, which claims the benefit of U.S. Provisional Application No. 63/224,210, filed Jul. 21, 2021, the entire contents of which are hereby incorporated by reference in its entirety.

### BACKGROUND

Embodiments of the present disclosure relate to handling of biological datasets, and more specifically, to systems of preprocessors to harmonize disparate ‘omics datasets by addressing bias and/or batch effects.

### BRIEF SUMMARY

According to embodiments of the present disclosure, methods of and computer program products for harmonizing a plurality of datasets are provided. A library comprising a plurality of preprocessors is read. Each preprocessor has an associated bias modality and is configured to map its bias modality to a common data space. An input dataset is read. A bias modality of the input dataset is determined. A preprocessor is selected from the library. The preprocessor corresponds to the bias modality of the input dataset. The preprocessor is applied to the input dataset to generate a harmonized dataset in the common data space.

According to embodiments of the present disclosure, methods of and computer program products for generating a library of preprocessors are provided. An input dataset is read. A library comprising a plurality of preprocessors is read. Each preprocessor has an associated bias modality and is configured to map its bias modality to a common data space. The input dataset is compared to each bias modality associated with the plurality of preprocessors, and it is determined thereby that the library does not include a preprocessor with an associated bias modality corresponding to the input dataset. A preprocessor is defined that is configured to map the input dataset to the common data space. The preprocessor is added to the library.

According to embodiments of the present disclosure, methods of and computer program products for training a classifier are provided. A library comprising a plurality of preprocessors is read. Each preprocessor has an associated bias modality and is configured to map its bias modality to a common data space. A plurality of input datasets is read. A bias modality of each of the plurality of input datasets is determined. One of the preprocessors from the library is applied to each of the plurality of input datasets to generate a plurality of harmonized datasets in the common data space. Each one of the preprocessors corresponds to the bias modality of its respective input dataset. The plurality of harmonized datasets is merged into a merged dataset in the common data space. A classifier is trained using the merged dataset.

In various embodiments, the input dataset comprises ‘omics data. In various embodiments, each of the plurality of input datasets comprises ‘omics data.

In various embodiments, each bias modality corresponds to an assay platform.

In various embodiments, each bias modality corresponds to a cancer type.

In various embodiments, selecting the preprocessor comprises performing a PCA, UMAP, t-SNE, or K-S test analysis using the input dataset. In various embodiments, comparing the input dataset to each bias modality comprises performing a PCA, UMAP, t-SNE, or K-S test analysis.

In various embodiments, each preprocessor is configured to apply quantile normalization, remove unwanted variation (RAV), ComBat, ComBat-Seq, BUS, BUS-Seq, or SVA.

### BRIEF DESCRIPTION OF THE SEVERAL VIEWS OF THE DRAWINGS

FIG. 1 is a schematic diagram of a system for harmonizing disparate ‘omics datasets according to embodiments of the present disclosure.

FIG. 2 is a flowchart illustrating modality definition according to embodiments of the present disclosure.

FIG. 3 is a scatter plot illustrating the separation of multiple datasets according to embodiments of the present disclosure.

FIG. 4 is a flowchart illustrating a method of fitting preprocessors for each bias modality according to embodiments of the present disclosure.

FIG. 5 is a scatter plot illustrating rectification of multiple datasets according to embodiments of the present disclosure.

FIG. 6 is a flowchart illustrating a method of fitting additional preprocessors according to embodiments of the present disclosure.

FIG. 7 is a flowchart illustrating a method of training a classifier according to embodiments of the present disclosure.

FIG. 8 is a flowchart illustrating a method of testing a classifier according to embodiments of the present disclosure.

FIG. 9 is a flowchart illustrating a method of evaluating a classifier according to embodiments of the present disclosure.

FIG. 10 is a flowchart illustrating a method of employing a classifier in a production environment according to embodiments of the present disclosure.

FIG. 11 is a flowchart illustrating a method of harmonizing a plurality of datasets according to embodiments of the present disclosure.

FIG. 12 is a flowchart illustrating a method of generating a library of preprocessors according to embodiments of the present disclosure.

FIG. 13 is a flowchart illustrating a method of training a classifier according to embodiments of the present disclosure.

FIG. 14 is a schematic diagram of a systems of preprocessors integrated with a data management system according to embodiments of the present disclosure.

FIG. 15 depicts a computing node according to embodiments of the present disclosure.

### DETAILED DESCRIPTION

Machine learning models may be trained on a variety of biological data to recognize a variety of conditions in a subject. However, different data sets, even those relevant to a single condition, may have significantly different statistical distributions. Any trained model implicitly assumes that new data, based on which it makes predictions, derive from the same distribution of data as the training set. This assumption is a faulty one, especially when training datasets are small, and/or arise from bias-prone sources, such as human tissue.

One reason that so few biomarkers, especially complex gene signatures, achieve clinical utility is because there are no good solutions to ensuring each new patient can be confidently assigned model predictions. This is because nearly every new data point carries some amount of bias that puts it outside the distribution of the training set.

One approach is to use a simple statistical model, such as a Z-score, to align the new data with the training distribution—but this would require recomputing the entire distribution including the new patient, and is thus not suitable for clinical applications where the model needs to be locked. Alternative approaches require retraining the model, and thus entail laborious validation of the updated model every time.

Sources of bias and misaligned data distributions constitute a major limitation to the development of models that work across diseases. Some models may learn the signal specific to one disease, so other disease samples would inherently fall outside that distribution. However, given a feature set that is applicable to multiple diseases, and a training set that has been normalized to a distribution inclusive of multiple diseases, the remaining challenge is to normalize each new patient to fit into that distribution. Thus a function is needed to map each new patient sample or group of patient samples back to the training distribution based on the relevant sources of bias.

Similarly, computational models often fail on new patients or biological samples due to technical bias introduced by assay type or sample handling. As with disease type, one must account for sources of technical bias. Similar discordance in dataset distributions arise when attempting to bridge from preclinical or translational experimental systems, such as cell lines or mice, into human patient datasets. Thus, a model trained on a preclinical dataset cannot serve to make predictions on a clinical dataset unless the clinical data are appropriately mapped to the model's training set distribution.

It will be appreciated from the forgoing that there is a need in the art for methods to map each new data point (or dataset), often representing a patient (or patient cohort), to the relevant training data distribution.

In particular, there is a need for methods enabling a single predictive model, already trained and with the algorithm locked, to work across different disease areas, patient groups, technical assay types, etc. Such models may consist of a machine learning algorithm like a neural network, random forest, logistic regression, etc. Each patient sample derives from some combination of a disease (or normal) tissue, that patient's demographic/geographic/ethnic background, and the various technical processes applied to the tissue sample to generate the input. All of these factors contribute bias to the sample data. The model needs to reliably predict some target variable (such as a clinical endpoint like tumor response or survival) on each patient sample regardless of those (or other) sources of bias.

As set out below, the present disclosure addresses these and other shortcomings of alternative approaches by providing a preprocessor library that streamlines validations by allowing the trained model to remain locked, with additional validations required only for new preprocessor functions. It will be appreciated that while the present disclosure is described in connection with ‘omics datasets (e.g., genomics, proteomics, metabolomics, metagenomics and transcriptomics), it is applicable to additional datasets in which data distribution requires rectification for model consistency.

As used herein, a preprocessor refers to a function that maps the distribution of one dataset onto the distribution of a training dataset. A preprocessor library as described herein is a collection of preprocessor functions that each have learned to map samples with certain types of bias to a training dataset distribution. The types of bias stem from, for example, tissue type, disease site of origin, patient demographic, technical assay (e.g., RNA-seq versus microarray), and other sources that will be appreciated by one of skill in the art.

In one exemplary embodiment, a library is provided for a biomarker algorithm (a neural network) that consists of preprocessors for datasets originating from gastrointestinal or reproductive tissues. This library will grow to include preprocessors for skin, lung, bone marrow and other tissue sites. The library includes other preprocessor functions for different kinds of technical assay, namely microarray and various flavors of RNA-sequencing sample prep.

Each preprocessor function is associated with a distribution characteristic of a specific bias modality, or sources of bias. The function enables new samples to be normalized. The data distributions may represent gene expression values, e.g., from total RNA-sequencing, mRNA-sequencing, microarray, etc. However, the distributions could also represent protein expression data, or any other quantitative measurement of a class of biological analytes.

In various embodiments, the library of preprocessors is dynamic, in that new samples can be automatically surveyed to determine which preprocessor will work best to map that sample's data to the training distribution. The library may be automated to detect the source(s) of bias, or may be controlled by user inputs specifying the various sources of bias. The number of preprocessors, and the sources of bias each represents, may change over time as more samples are analyzed and mapping functions are optimized.

One advantage of these methods is that they allow a trained predictive model to remain locked for the sake of clinical applications or analytical consistency, while also enabling the inclusion of virtually any new patient sample in the analytical workflow. Because each preprocessor achieves the same goal—normalization to a previously defined distribution—this step in a clinical application workflow can be included so long as the criteria for successful normalization and contingencies for failed normalization are pre-defined. This is more favorable than alternative approaches, such as those relying on a Z-score statistic, where any new sample requires re-training the model on a new distribution.

Another advantage of the methods provided herein is that they enable aligning multiple datasets such that they can be used together either in training or testing a model.

Thus, the size of the training set can be increased, avoiding a common limitation in building biological models.

Systems of preprocessors as set out herein have several advantages over alternative approaches, enabling:
1. Application of ML models across different diseases, e.g., different cancer types;
2. Application of ML models across datasets derived from different assay platforms, e.g., RNA-seq vs. microarray;
3. Application of ML models across datasets harboring other modalities of bias, e.g., clinical lab, testing location, sequencing facility;
4. Application of ML models across R&D stages, e.g., model trained on preclinical data applied to clinical data; and
5. Implementation of pan-disease (machine learning) models in regulated (clinical) environment.

This is achieved by decoupling the step of normalizing training or evaluation data from the steps of training or evaluating a model. A dynamic resource of normalization functions (preprocessors) is thus provided, corresponding to any number of bias modalities that have been defined for each relevant data space. This enables the application of a model across datasets derived from sources of various biases.

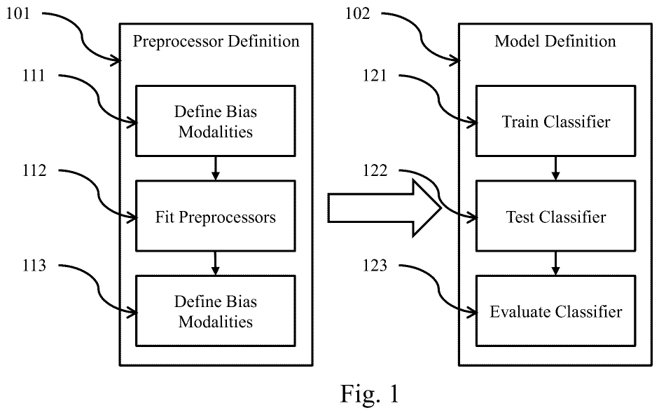

Referring to FIG. 1, the overall stages of a process according to the present disclosure are illustrated. At 101, preprocessors are defined. At 102, models (e.g., predictive models) are defined. During preprocessor definition 101, bias modalities are defined at 111, preprocessors are fit at 112, and additional preprocessors are fit at 113, each of which are detailed further below. During model definition 102, a classifier is trained at 121, tested at 122, and evaluated at 123, each of which are detailed further below.

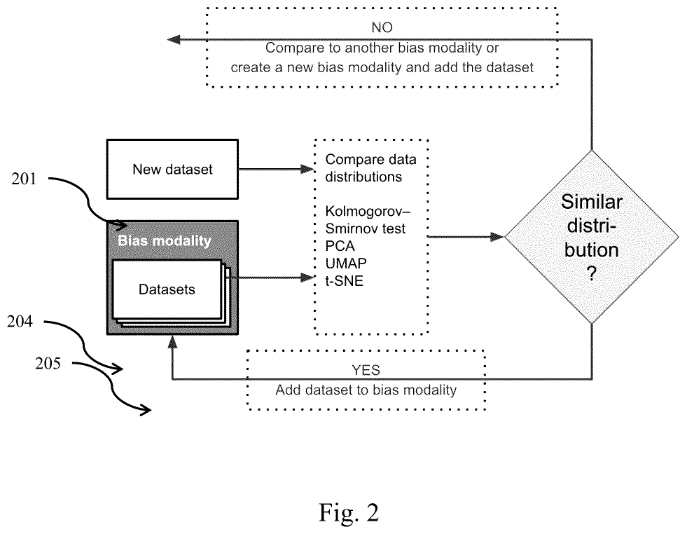

Referring to FIG. 2, bias modality definition is illustrated. Each new dataset 201 is compared 202 to existing datasets 203 associated with a given bias modality 204. In particular, the data distributions of available datasets, including data intended to be training data and evaluation data, are compared. Bias modality may be inferred from known differences between the datasets, e.g., metadata such as tissue of origin or technical assay platform, based on which of these contribute to separation of the data distributions. Separation of dataset distributions may be analyzed by PCA, UMAP, t-SNE, K-S tests, or other methods known in the art. In some embodiments, either metadata or statistical comparators are employed, while in other embodiments a combination of these factors are considered. For example, a same tissue type and a similar statistical distribution may be required to create an association with a given bias modality.

In various embodiments, each new dataset is compared to multiple datasets associated with the bias modality. In some embodiments, metadata about each bias modality is stored in order to more efficiently perform the comparison step. In such embodiments, the metadata may vary according the preprocessing method. For example, if the preprocessor is a standard scaler, the mean and standard deviation for a given modality may be stored. More complex preprocessors may require more metadata. Generally, the metadata captures a complete state of a preprocessor and defines the preprocessor in full.

In various embodiments, the initial dataset for a given bias modality is definitional. That is, subsequent data are mapped to the data distribution of the initial dataset. It will be appreciated that bias modality definition is separate from model training. In the case of a meta-training set, after modalities are identified, the datasets are mapped to the same distribution to define the meta-training set prior to model definition.

Where the distribution of a new dataset is similar to a known bias modality, the dataset is associated with that bias modality. Where the distribution of a new dataset is not similar to a known bias modality, it is compared to another bias modality or, if there are none, a new bias modality is defined and associated with the new dataset.

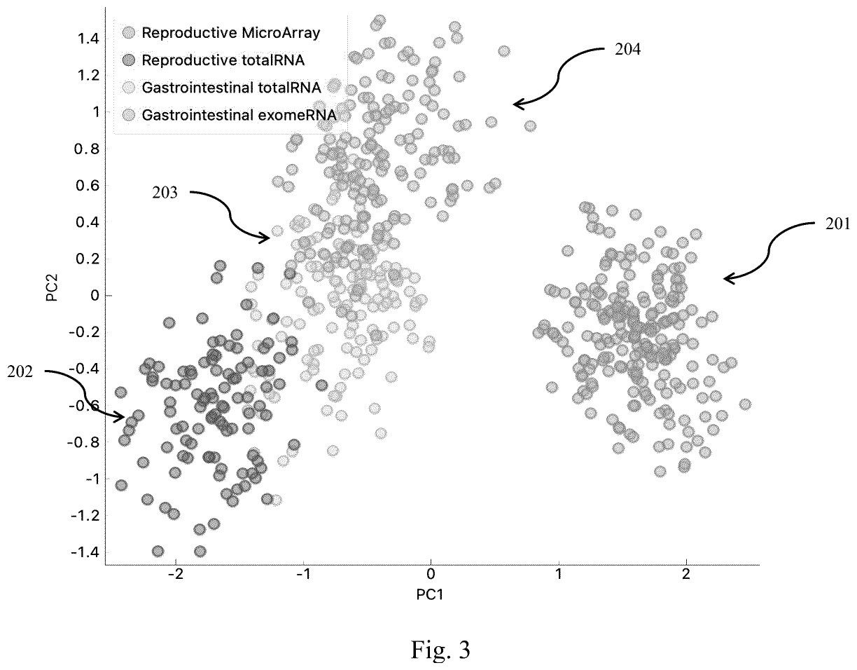

Referring to FIG. 3, a scatter plot is provided illustrating the separation of multiple datasets. Reproductive MicroArray corresponds to cluster 201; Reproductive totalRNA corresponds to cluster 202; Gastrointestinal totalRNA corresponds to cluster 203; and Gastrointestinal exomeRNA corresponds to cluster 204. As shown, technical assay platform (microarray versus RNAseq) represents the difference on the x-axis (PC1), while tissue of origin (gastrointestinal versus reproductive) represents the main delta on the y-axis (PC2). Within like-tissue types on the Y-axis, further separation is observed based on the specific type of data generation method employed (total-RNAseq versus exome-RNAseq).

Each dataset presenting a unique distribution may contribute a new bias modality. Datasets for which a bias modality has already been identified may be added to that bias modality.

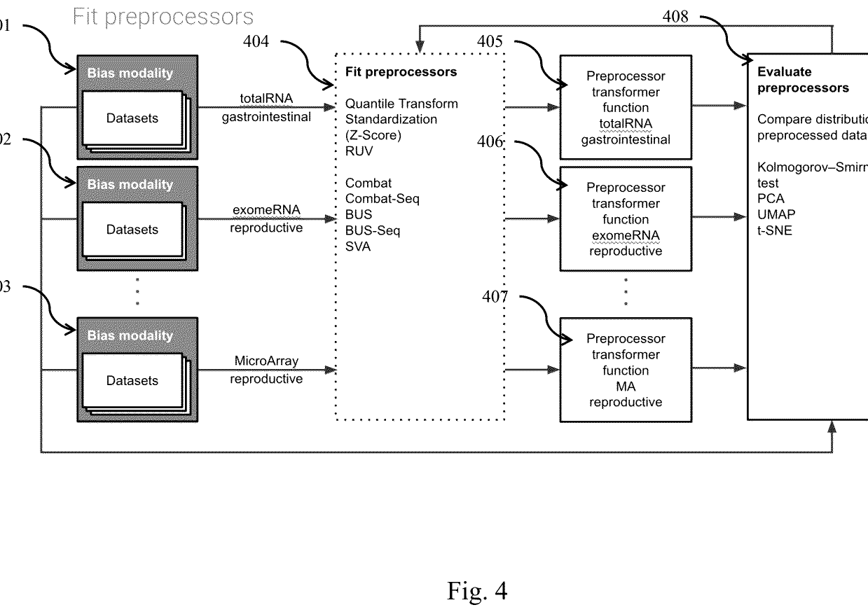

Referring to FIG. 4, the processes of fitting preprocessors for each bias modality is illustrated. In this example, three exemplary bias modalities are shown: gastrointestinal totalRNA 401; reproductive exomeRNA 402; and reproductive MicroArray 403.

Each of a plurality of preprocessors is fit 404 for each bias modality 401 . . . 403. The preprocessor is applied to all datasets within that bias modality. Preprocessors may be statistical functions such as Quantile Normalization or Standardization (e.g., Z-score), or more elaborate methods like RUV (remove unwanted variation), ComBat, ComBat-Seq, BUS, BUS-Seq, SVA, etc. In various embodiments, the same type of function is applied to each bias modality and all datasets for a given preprocessor library. The function is tailored to fit to the given bias modality individually. The resulting preprocessor transformer functions 405 . . . 407 are then associated with the respective bias modality. Those functions are evaluated 408 in much the same way the biases were originally identified, to determine if the datasets now align irrespective of their original bias modality.

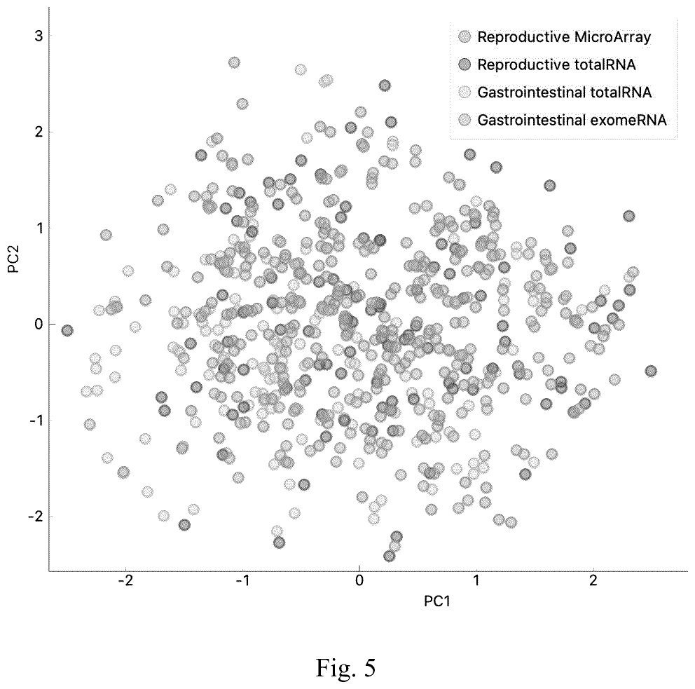

Referring to FIG. 5, a scatter plot is provided illustrating the rectification of the multiple datasets of FIG. 3.

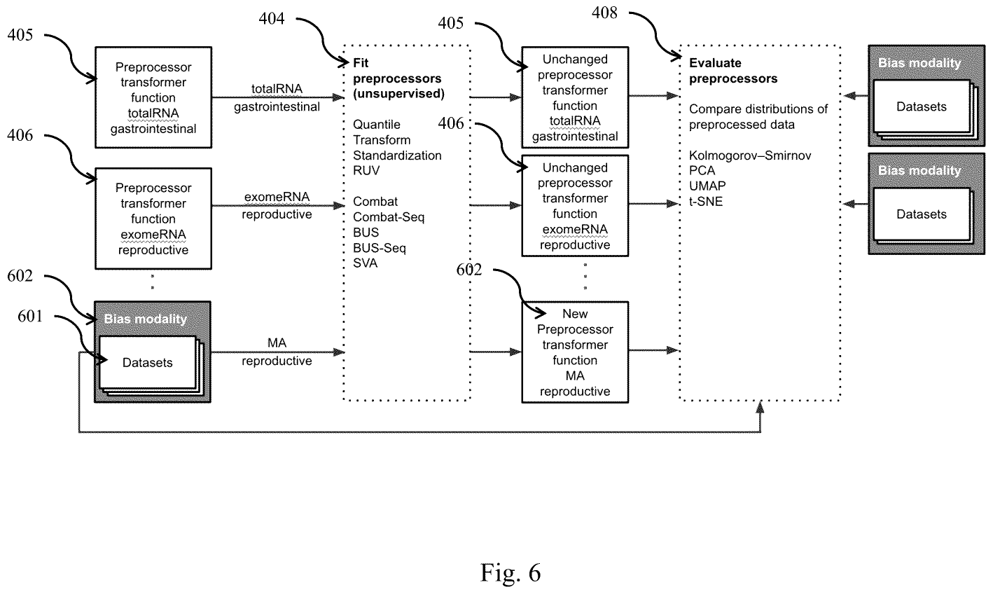

Referring to FIG. 6, a process for fitting additional preprocessors is illustrated. As new datasets are identified or generated to be analyzed by the model, these may either conform to an existing bias modality, or represent a new bias modality. In the former case, they may be transformed with an existing preprocessor function. In the event that a new bias modality is identified, the data distribution of that bias modality is compared to the aligned distribution of the prior transformed datasets.

In this example, new datasets 601 are associated with new bias modality 602. As discussed above with regard to FIG. 4, a new preprocessor function is then fit at 404 to the new bias modality 602. This new preprocessor function 602 is then added to the library of preprocessors.

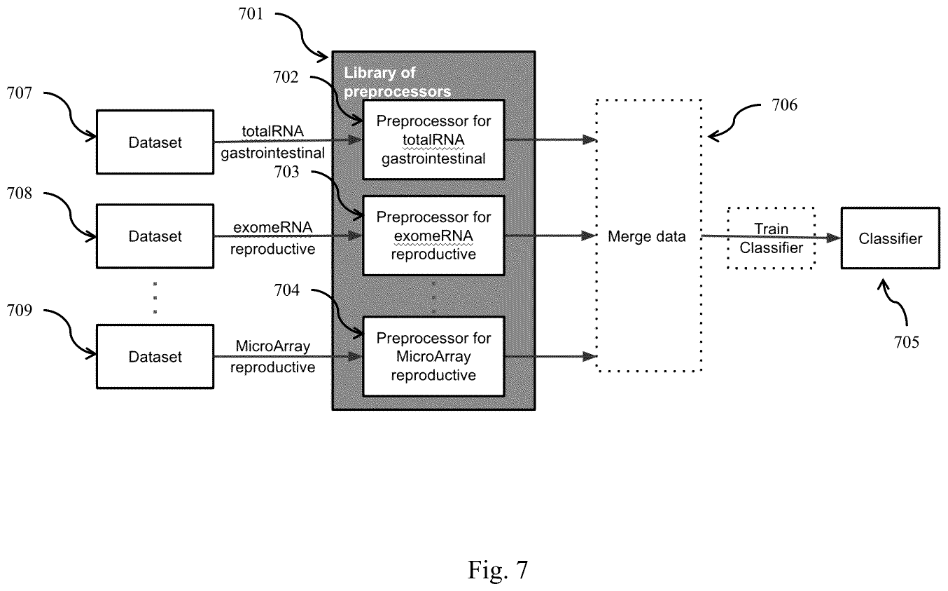

Referring to FIG. 7, methods of training a classifier according to embodiments of the present disclosure are illustrated. A library 701 of preprocessors 702 . . . 704 is assembled as set forth above. This enables a classifier 705 to be trained on merged dataset 706 in addition to a single dataset. In either case, the one or more input datasets 707 . . . 709 are transformed by a preprocessor function 702 . . . 704 prior to training. The preprocessor library 701 enables the construction of a merged (or meta-) dataset 706 from otherwise disparate and non-interoperable individual datasets. Each input dataset 707 . . . 709 to the meta-dataset 706 is transformed using the preprocessor function corresponding to the respective bias modalities, mapping each input dataset to a shared data space suitable for model training.

It will be appreciated that the methods described herein are usable with any classifier known in the art. Examples of suitable classifiers include random decision forests, linear classifiers, support vector machines (SVM), and neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

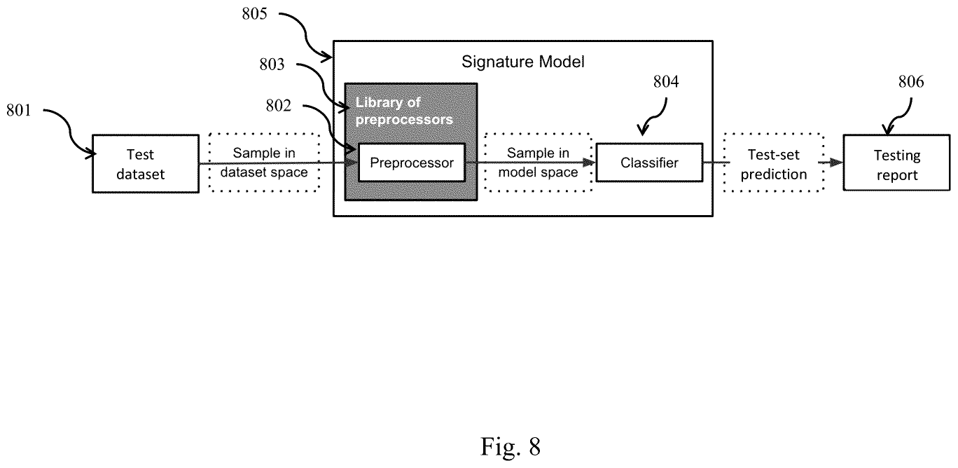

Referring to FIG. 8, methods of testing a classifier are illustrated. A test dataset 801 may consist of measurements from one or more patients, patient samples, or experimental specimens (for example, gene expression values from a cohort of patients in a clinical trial). Each sample resides in the test dataset space. The test dataset space corresponds to the distribution of that particular data set, without further rectification. These data are transformed using the preprocessor function 802 for the appropriate bias modality to map each sample into the model space. The preprocessor function is selected from the library of preprocessors 803. The model space corresponds to the universal distribution to which all bias modalities are mapped, as described further above. The data then may be fed into a classifier 804 for classification. The classifier 804 outputs some test-set prediction (e.g., a therapeutic outcome, drug response, disease phenotype, etc.), which forms the basis for determining/reporting on the model performance.

Correctively, the library of preprocessors 803 and classifier 804 are referred to as signature model 805. In various embodiments, a testing report 806 may be provided including the test-set prediction from classifier 804.

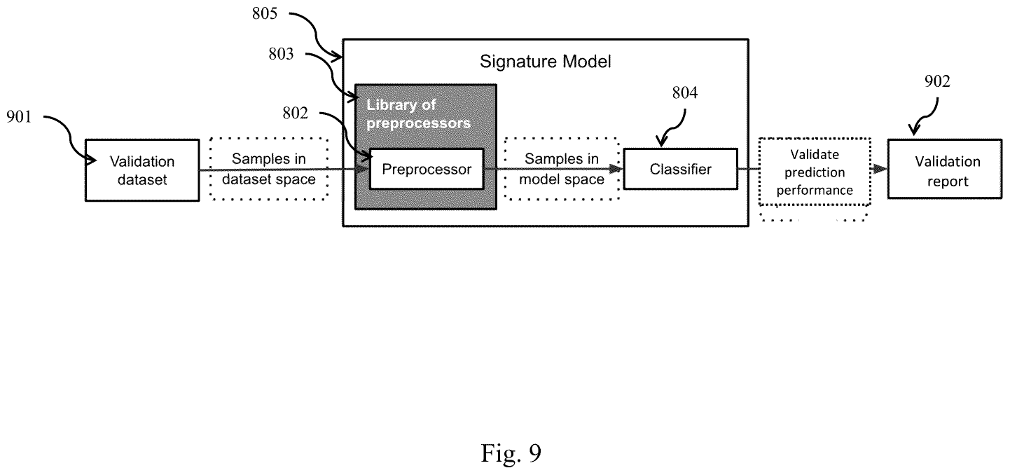

Referring to FIG. 9, methods of evaluating a classifier are illustrated. The process described above in connection with FIG. 8 for test data is repeated for each validation dataset 901, as well as for future datasets arising from commercial or real world applications of the model. The resulting predictions are used to determine model performance according to methods known in the art and are given in a validation report 902.

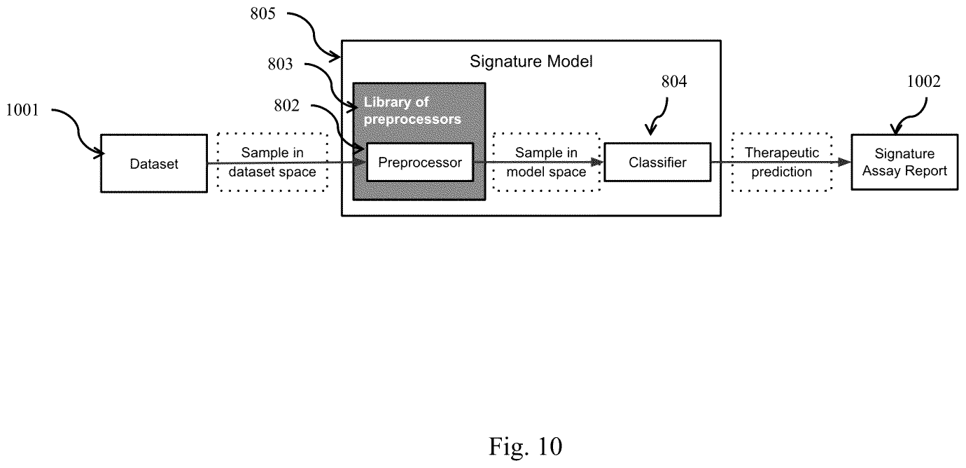

Referring to FIG. 10, methods of employing a classifier in a production environment are illustrated. The process described above in connection with FIGS. 8-9 for test and validation data is repeated for each dataset 1001, as well as for future datasets arising from commercial or real world applications of the model. The resulting therapeutic predictions are used to generate a signature assay report 1002 according to methods and including relevant clinical results known in the art.

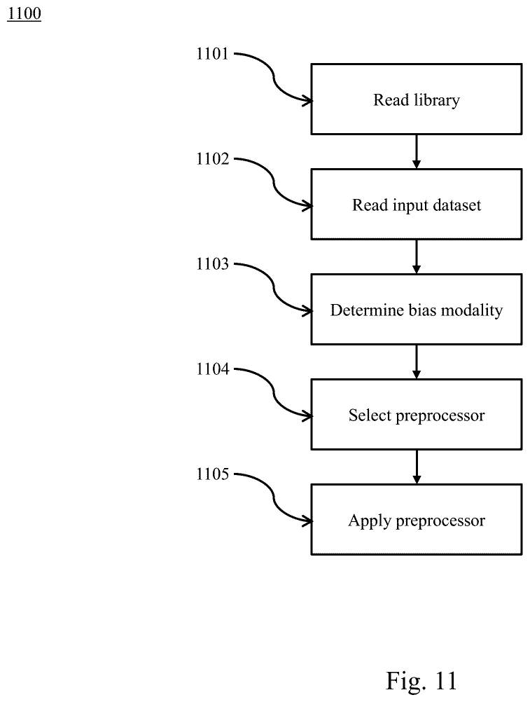

With reference now to FIG. 11, methods of harmonizing a plurality of datasets are illustrated. At 1101, a library comprising a plurality of preprocessors is read. Each preprocessor has an associated bias modality and is configured to map its bias modality to a common data space. At 1102, an input dataset is read. At 1103, a bias modality of the input dataset is determined. At 1104, a preprocessor is selected from the library. The preprocessor corresponds to the bias modality of the input dataset. At 1105, the preprocessor is applied to the input dataset to generate a harmonized dataset in the common data space.

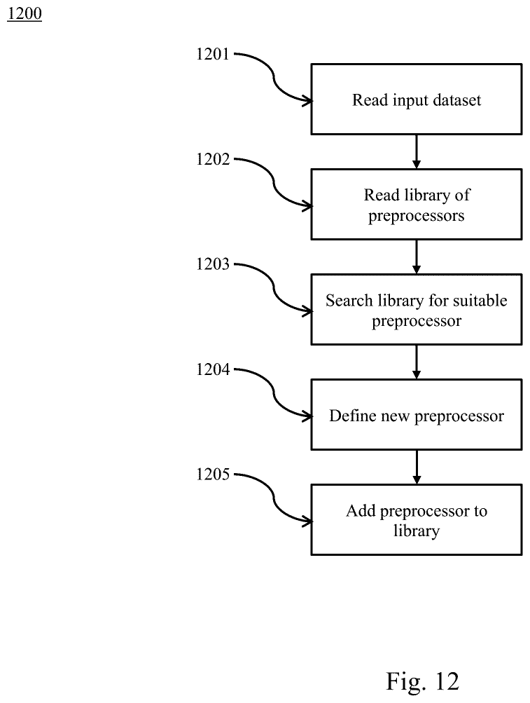

With reference now to FIG. 12, methods of generating a library of preprocessors are illustrated. At 1201, an input dataset is read. At 1202, a library comprising a plurality of preprocessors is read. Each preprocessor has an associated bias modality and is configured to map its bias modality to a common data space. At 1203, the input dataset is compared to each bias modality associated with the plurality of preprocessors, and it is determined thereby that the library does not include a preprocessor with an associated bias modality corresponding to the input dataset. At 1204, a preprocessor is defined that is configured to map the input dataset to the common data space. At 1205, the preprocessor is added to the library.

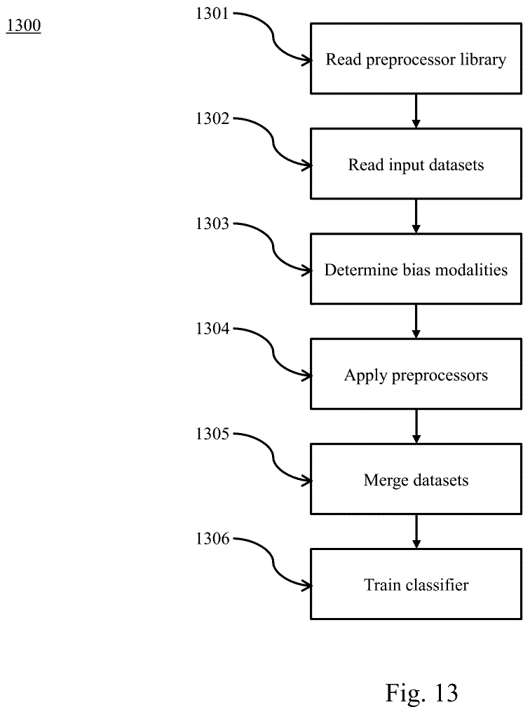

With reference now to FIG. 13, methods of training a classifier are illustrated. At 1301, a library comprising a plurality of preprocessors is read. Each preprocessor has an associated bias modality and is configured to map its bias modality to a common data space. At 1302, a plurality of input datasets is read. At 1303, a bias modality of each of the plurality of input datasets is determined. At 1304, one of the preprocessors from the library is applied to each of the plurality of input datasets to generate a plurality of harmonized datasets in the common data space. Each one of the preprocessors corresponds to the bias modality of its respective input dataset. At 1305, the plurality of harmonized datasets is merged into a merged dataset in the common data space. At 1306, a classifier is trained using the merged dataset.

### Additional Examples

In addition to the examples provided above, preprocessors may be defined and/or trained to distinguish between different bias modalities. Exemplary preprocessor types include: multi-tissue, single origin tissue, Balanced Upsampled, Unbalanced, Multi-omic, Omic-platform specific (DNA<>RNA<>Protein; Single cell<>bulk sequencing; Spatial<>1D; Sample handling (FFPE<>Fresh frozen<>Aspirate<>archival; Microdissection<>scrape-all); Tissue compartment (cell free (circulating)<>tumor<>normal; Primary<>metastatic); Disease model (e.g., tumor<>cell line<>mouse model<>organoid); Demographic (e.g., based on geography, ethnicity, age, gender); Clinical (line of therapy; disease stage; treatment history, e.g., naive<>refractory).

Detailed descriptions of some of these bias modalities are provided below. However, it will be appreciated that preprocessor systems according to the present disclosure are applicable to the various bias modalities described above, as well as many others known in the art.

In a first example a Platform/Chemistry Preprocessor is provided. A Platform/Chemistry Preprocessor is a mapping function that serves to harmonize data measuring the same type of analyte (e.g., RNA abundance or DNA mutations) using different sample preparation chemistries or sequencing platforms. If a model is trained, for example, on gene expression data from a microarray platform, as with the Xerna TME Panel, a Platform Preprocessor will be used for input data derived from RNA-sequencing. Even when data of the same type derive from the same sequencing platform, e.g., an Illumina HiSeq machine, the tissue samples may be processed using distinct chemistries such as total RNA versus mRNA-enriched, which can create a bias modality. An analogous example would be DNA variants measured by a targeted gene panel versus whole exome sequencing.

Such a preprocessor is particularly useful where a model has been trained on historical data from an obsolete microarray platform. The clinical trial assay, however, may use total RNA-sequencing while the commercial RUO diagnostic kit will use mRNA-sequencing, and the companion diagnostic test will use targeted gene sequencing. To facilitate the clinical and commercial development of this model into a regulated device, a Platform/Chemistry Preprocessor is needed.

In a second example, a Multi-Tissue Preprocessor is provided. A multi-tissue preprocessor is one in which the mapping function is trained on input data from various source tissues (ideally in roughly equal proportions). The inclusion of more than one tissue allows for a greater portion of the data landscape to be captured due to the inherent biological heterogeneity when comparing samples from different tissues of the body. As an example, a liver cancer sample is different in both expression values and phenotype to a tumor from another region such as the brain. Including both of these tissue types in the preprocessor enables the downstream model to account for tissue-specific differences in producing comparable outputs. For instance, the outputs might directly compare the immunogenicity or angiogenicity of liver versus brain tissue. A greater number of tissue types, particularly around the edges of the phenotypic data space, may result in improved predictive performance of the model.

This type of preprocessor may be desirable if the downstream model/device is intended to be pan-tissue/pan-disease, and a consistent technology platform/chemistry will be used for all data generation. Such a preprocessor may also be helpful if the device purpose is to guide selection of different disease indications for treatment.

In an example embodiment, the Xerna TME Panel outputs four disease subtypes based on the intersection of immunogenic and angiogenic signal learned by the model. A multi-tissue preprocessor provides context about the underlying biology of the tissue type relative to the other tissue types in the data space. Eighteen different tissue types from the TCGA were used to develop a multi-tissue preprocessor. The mean angiogenic and immunogenic scores were computed when these samples were run through the model to understand the relative position of patient samples based on their tissue of origin.

In a third example, a Single-Tissue Preprocessor is provided. A single-tissue preprocessor can be configured with samples collected from a single tissue type, so long as sufficient numbers of samples (n=>75) are analyzed to ensure the data space is appropriately mapped. The single tissue preprocessor allows samples derived from the same tissue type to be compared relative to each other without introducing heterogeneity and biological variability arising from tissue source.

Such a preprocessor is useful in the context of clinical trials. Clinical trials are generally conducted on a specific indication, which includes the primary location of the tumor. Thus, data is generated on a single tissue basis. These clinical trial data are generally generated on the same platform, thereby constraining the observed batch effect. Furthermore, companion diagnostic devices are indication (tissue) specific. Thus, for a model that is incorporated into a companion diagnostic, a single-tissue preprocessor is appropriate.

In an exemplary embodiment the Xerna TME Panel provides a companion diagnostic device for the use of the drug navicixizumab in ovarian cancer. A single-tissue preprocessor specific to ovarian (or more generally gynecological) cancer tissue was developed to support data handling for analysis by this device.

In a fourth example, a Balanced Preprocessor is provided. An uneven distribution of samples from different bias modalities can make achieving a harmonized data space challenging. Samples from under-represented bias modalities may be replicated (upsampled) in silico to a common multiple in order to boost the overall number of samples and minimize the relative differences between bias modalities prior to training the preprocessor function. For example, consider the development of a multi-tissue preprocessor, which requires a sufficiently large number of samples roughly equally distributed among each of the input tissue types. In practice, samples across different tissue types are available in varied quantities. If a preprocessor is to be developed on multiple tissue types with a different number of samples per tissue, bias will be introduced towards the over-represented tissues. Upsampling, or the digital replication of samples for each tissue type, limits bias introduced by an uneven distribution of samples per tissue type. In additional embodiments, upsampling is provided by generation of digital replicates created de novo, by simulating observed variation in expression profiles in general or in particular tissues.

Such a preprocessor is useful in cases where samples are generally not available in sufficient quantities to otherwise develop a robust preprocessor. Upsampling through in silico replication is a means of achieving sufficient numbers of data points to train a useful preprocessor.

In a fifth example, a Multi-Omic Preprocessor is provided. Data used for model training and model predictions may derive from any number of ‘omics analysis types. These include but are not limited to data representing the genome, proteome, transcriptome, metabolome, and epigenome. Omics data may be generated through bulk collection of cells/tissue, or from physically separated single cells. Further, omics data may be measured as a linear sequence, a two dimensional matrix, or in three dimensions including relative spatial positions.

Some models may take as inputs a combination of ‘omics data that need to be transformed to a uniform data space prior to training and/or classification. A multi-omic preprocessor is a mapping function trained to harmonize data collected from different ‘omes.

Such a preprocessor is useful for classification of tissues and bodily fluids with high sensitivity and specificity that benefits from multi-omic data. Certain analytics platforms, e.g., Mission Bio Tapestri and Codetta Biosciences, routinely measure DNA, RNA and protein from the same samples. Modeling all of these ‘omes in concert requires relating the values of all the ‘omes within a specimen. Thus a multi-omic preprocessor is useful for models that rely on integrated data.

In a sixth sample, a Bulk v. Single Cell preprocessor is provided. In bulk sequencing, the target molecule is extracted from a collection of cells or tissue which will likely be composed of multiple cell-types. In single cell sequencing, cells are physically separated prior to sequencing. Sometimes single cells are classified based on various attributes like cell surface markers prior to sequencing. There are differences in the output when comparing the respective techniques. Bulk sequencing provides a much broader picture as to the entire tissue sample, while single cell provides information directly about subgroups or types of cells of interest. Bulk sequencing is advantageous in that the data are less noisy or heterogeneous than a single cell, however bulk has the disadvantage of averaging out the signal that may be biologically interesting between different cells. scRNA sequencing data is sparse, posing a particular problem with mapping to bulk RNA-Seq.

Such a preprocessor is useful where one wishes to map between single-cell and bulk sequencing data. The goal may be to infer a set of features from bulk sequencing based on relative values of those features in a single cell context. Bulk sequencing is cheaper, more widely available, and more reproducible than single cell, thus today bulk sequencing is preferred as a clinical tool in a regulated environment. Nevertheless, one may wish to train a model on single cell data, but then use that model to analyze clinical data generated by bulk sequencing.

In a seventh example, a Sample Handling preprocessor is provided. A Sample Handling Preprocessor is trained to mitigate the batch effect introduced when tissue samples are handled differently prior to data generation. Bias modalities may be associated with any number of tissue collection, storage and processing methods. In the cancer diagnostics space, for example, samples may come from fresh frozen biopsy, biopsy aspirate, core needle biopsy, formalin fixed paraffin embedded (FFPE) slides, etc. The duration of time the samples have been stored, e.g., fresh vs. archival, introduces bias. Further, the manner in which tissue has been collected for extraction and sequencing, such as microdissection vs scrape all, introduces bias.

Such a preprocessor is useful where a model is developed for use in a clinical trial assay (CTA), such as the Xerna TME Panel. The analytical validation studies for the CTA may use archival FFPE tissue, while the intended use during the clinical trial may be fresh frozen biopsy. A Sample Handling preprocessor would enable the trained model to be deployed for both the analytical validation and clinical trial use.

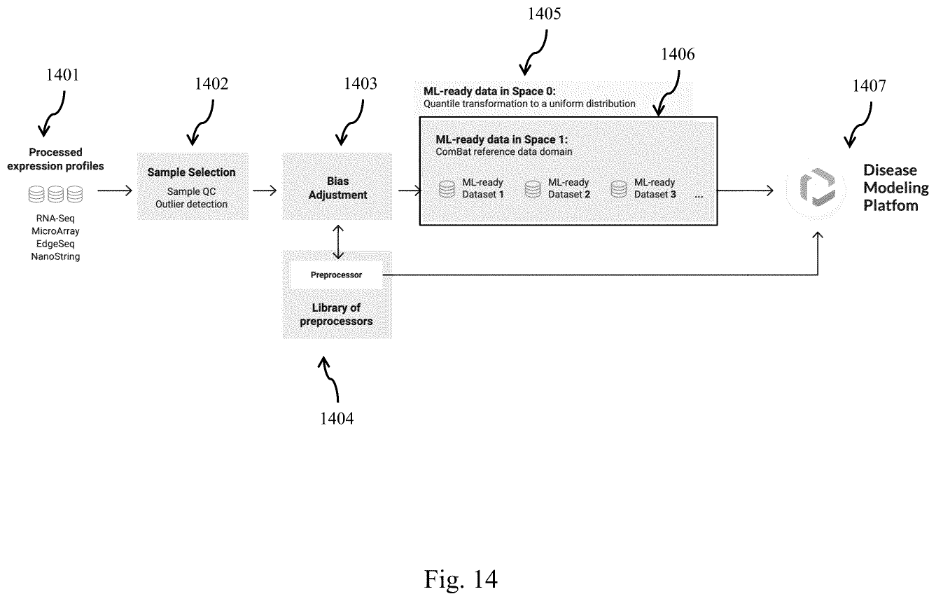

Referring now to FIG. 14, systems of preprocessors as set out herein may be integrated with a data management system to create a data flywheel. The data flywheel increases the momentum of data products such as clinical biomarkers at an accelerating rate due to the strategic use and reuse of curated, annotated, and bias-adjusted data collections.

FIG. 14 illustrates the integration of the systems of preprocessors with an exemplary data management system, Expressions by Genialis. Distinct datasets are transformed into a common data plane using the system of preprocessors. Genialis Expressions extends data management capabilities to all versions of transformed data (also called ML-ready data) that are managed in the same manner as the original data.

In particular, processed expression profiles 1401 may include a variety of data sources such as RNA-Seq, MicroArray, EdgeSeq, or NanoString. Samples selection 1402 may include quality control of the samples as well as outlier detection and other data cleanup tasks. Bias adjustment 1403 applies the library of preprocessors 1404 as described herein. The resulting adjusted data may be provided in a plurality of data spaces 1405 . . . 1406 and in turn provided to a disease modeling platform 1407.

This data flywheel architecture enables the re-use of datasets such that every new dataset increases the potential of the entire system to generate value. Biomarker models may thus be developed at an increasing pace over time.

In addition to the above use cases, the present disclosure may be applied in the context of federated learning. Federated learning is a learning paradigm seeking to address the problem of data governance and privacy by training algorithms collaboratively without exchanging the data itself. Biomedical ‘omics datasets used to train and validate biomarker models may be owned by distinct parties and deposited in their corresponding computational environments. A federated learning system may be employed to learn and validate biomarker models within the computational environments of the federation partners.

Partner datasets are subject to their unique biases. The system of preprocessors is a key technology to automate bias adjustment in distinct ‘omics datasets and can be integrated into federated learning training algorithms.

The data required to train and validate clinical biomarkers typically are owned by clinical research, diagnostic and pharmaceutical stakeholders. Each of these data owners is reluctant to share data with the competition. They may consider their proprietary data of utmost confidentiality and of high potential value. Further, they may be legally constrained in their rights to share the data. Thus, a federated learning system is one solution to overcome the hesitance or restrictions on data sharing for the sake of model development. Preprocessors enable sharing bias modalities across federation partners. Thus preprocessors are key to unlocking a potentially disruptive business approach.

It will be appreciated that preprocessors as described herein may be deployed in a variety of environments. For example, a preprocessor system may be deployed as a set of microservices. A microservices architecture is a design pattern in which each microservice is one small piece of a bigger overall system. Each microservice performs a specific and limited scope task that contributes to the end result. For example, APIs may be defined for tasks including “fit a preprocessor” or “identify the corresponding preprocessor from the library of preprocessors.” Microservices are independent workflows that communicate over well-defined APIs. The microservices architecture makes applications easier to scale and faster to develop.

In another example a preprocessor system may be deployed in a distributed architecture. A distributed system is a system whose components are located on different networked computers, which communicate and coordinate their actions by passing messages to one another from any system. The components interact with one another in order to achieve a common goal, in our case, to train a biomarker classifier on multiple decentralized servers, each holding part of the training data.

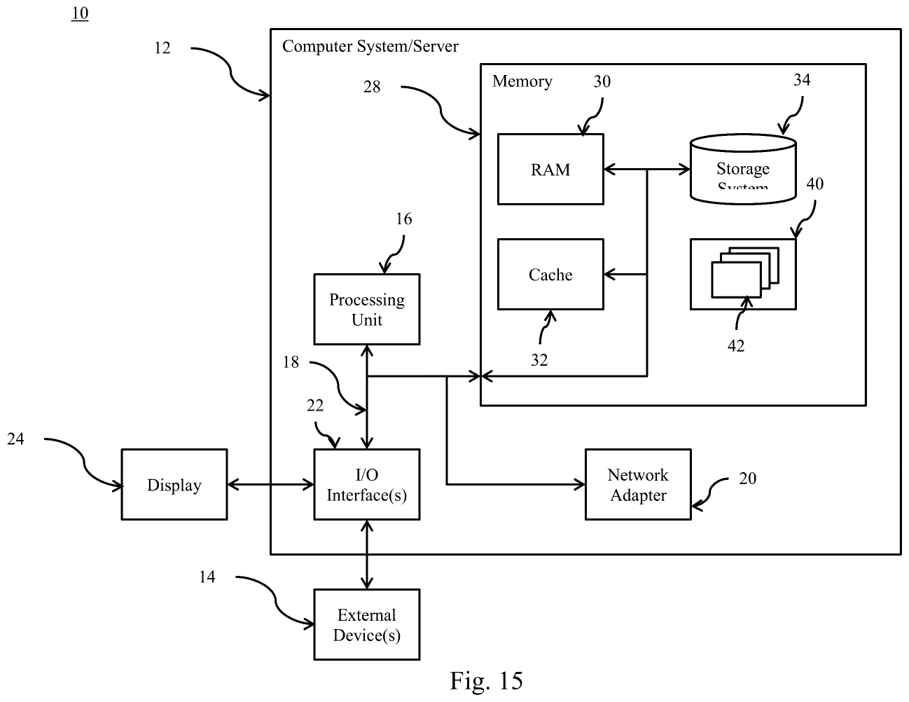

Referring now to FIG. 15, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 15, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of harmonizing a plurality of datasets, the method comprising:
reading a library comprising a plurality of preprocessors, each preprocessor having an associated bias modality and being configured to map its bias modality to a common data space;
reading an input dataset;
determining a bias modality of the input dataset;
selecting a preprocessor from the library, the preprocessor corresponding to the bias modality of the input dataset; and
applying the preprocessor to the input dataset to generate a harmonized dataset in the common data space.

2. The method of claim 1, wherein the input dataset comprises ‘omics data.

3. The method of claim 1, wherein each bias modality corresponds to an assay platform.

4. The method of claim 1, wherein each bias modality corresponds to a cancer type.

5. The method of claim 1, wherein selecting the preprocessor comprises performing a PCA, UMAP, t-SNE, or K-S test analysis using the input dataset.

6. The method of claim 1, wherein each preprocessor is configured to apply quantile normalization, remove unwanted variation (RAV), ComBat, ComBat-Seq, BUS, BUS-Seq, or SVA.

7. A method of generating a library of preprocessors, the method comprising:
reading an input dataset;
reading a library comprising a plurality of preprocessors, each preprocessor having an associated bias modality and being configured to map its bias modality to a common data space;
comparing the input dataset to each bias modality associated with the plurality of preprocessors, and determining thereby that the library does not include a preprocessor with an associated bias modality corresponding to the input dataset;
defining a preprocessor configured to map the input dataset to the common data space; and
adding the preprocessor to the library.

8. The method of claim 7, wherein the input dataset comprises ‘omics data.

9. The method of claim 7, wherein each bias modality corresponds to an assay platform.

10. The method of claim 7, wherein each bias modality corresponds to a cancer type.

11. The method of claim 7, wherein comparing the input dataset to each bias modality comprises performing a PCA, UMAP, t-SNE, or K-S test analysis.

12. The method of claim 7, wherein each preprocessor is configured to apply quantile normalization, remove unwanted variation (RAV), ComBat, ComBat-Seq, BUS, BUS-Seq, or SVA.

13. A method of training a classifier, the method comprising:
reading a library comprising a plurality of preprocessors, each preprocessor having an associated bias modality and being configured to map its bias modality to a common data space;
reading a plurality of input datasets;
determining a bias modality of each of the plurality of input datasets;
applying one of the preprocessors from the library to each of the plurality of input datasets, each one of the preprocessors corresponding to the bias modality of its respective input dataset to generate a plurality of harmonized datasets in the common data space;
merging the plurality of harmonized datasets into a merged dataset in the common data space; and
training a classifier using the merged dataset.

14. The method of claim 13, wherein each of the plurality of input datasets comprises ‘omics data.

15. The method of claim 13, wherein each bias modality corresponds to an assay platform.

16. The method of claim 13, wherein each bias modality corresponds to a cancer type.

17. The method of claim 13, wherein each preprocessor is configured to apply quantile normalization, remove unwanted variation (RAV), ComBat, ComBat-Seq, BUS, BUS-Seq, or SVA.

18. A computer program product for harmonizing a plurality of datasets, the computer program product comprising a computer readable storage medium having program instructions embodied therewith, the program instructions executable by a processor to cause the processor to perform a method comprising:
reading a library comprising a plurality of preprocessors, each preprocessor having an associated bias modality and being configured to map its bias modality to a common data space;
reading an input dataset;
determining a bias modality of the input dataset;
selecting a preprocessor from the library, the preprocessor corresponding to the bias modality of the input dataset; and
applying the preprocessor to the input dataset to generate a harmonized dataset in the common data space.

19. A computer program product for generating a library of preprocessors, the computer program product comprising a computer readable storage medium having program instructions embodied therewith, the program instructions executable by a processor to cause the processor to perform a method comprising:
reading an input dataset;
reading a library comprising a plurality of preprocessors, each preprocessor having an associated bias modality and being configured to map its bias modality to a common data space;
comparing the input dataset to each bias modality associated with the plurality of preprocessors, and determining thereby that the library does not include a preprocessor with an associated bias modality corresponding to the input dataset;
defining a preprocessor configured to map the input dataset to the common data space; and
adding the preprocessor to the library.

20. A computer program product for training a classifier, the computer program product comprising a computer readable storage medium having program instructions embodied therewith, the program instructions executable by a processor to cause the processor to perform a method comprising:
reading a library comprising a plurality of preprocessors, each preprocessor having an associated bias modality and being configured to map its bias modality to a common data space;
reading a plurality of input datasets;
determining a bias modality of each of the plurality of input datasets;
applying one of the preprocessors from the library to each of the plurality of input datasets, each one of the preprocessors corresponding to the bias modality of its respective input dataset to generate a plurality of harmonized datasets in the common data space;
merging the plurality of harmonized datasets into a merged dataset in the common data space; and
training a classifier using the merged dataset.
